SEQUENCE LISTING (14 sequences)

SEQ ID NO: 1 - protein, 14 aa, N.gonnorhoeae
GGEKAAEAPAAEAS

SEQ ID NO: 2 - protein, 88 aa, N.gonnorhoeae
MKKSLFAAALLSLALAACGGEKAAEAPAAEASSTEAPAAEAPAAEAPAAEAAAAEAPAAEAPAAEAPAAEAAATEAPAAEAPAAEAAK

SEQ ID NO: 3 - protein, 15 aa, N.gonnorhoeae
CGGEKAAEAPAAEAS

SEQ ID NO: 4 - protein, 5 aa, N.gonnorhoeae
AAEAS

SEQ ID NO: 5 - protein, 5 aa, N.gonnorhoeae
AAEAA

SEQ ID NO: 6 - protein, 5 aa, N.gonnorhoeae
AAEAP

SEQ ID NO: 7 - protein, 5 aa, N.gonnorhoeae
  VARIANT 5: any amino acid
AAEAX

SEQ ID NO: 8 - protein, 98 aa, N.meningitidis
MKKSLFAAALLSLVLAACGGEKAAEAPAAEAPAAEAPATEAPAAEAPAAEAPAAEAPAAEAAATEAPAAEAAATEAPAAEAAATEAPAAEAPAAEAAK

SEQ ID NO: 9 - protein, 14 aa, N.meningitidis
GGEKAAEAPAAEAP

SEQ ID NO: 10 - protein, 15 aa, N.meningitidis
CGGEKAAEAPAAEAP

SEQ ID NO: 11 - DNA, 552 bp, N.meningitidis
atgaaagcgtatctggctctgatttctgccgccgttatcggtttggctgcctgctctcaagaacctgccgcgcctgctgccgaggcaactcctgctgctgaagcacccgcttccgaagcgcctgccgccgaagctgctcctgcagatgctgccgaagcccctgctgccggcaactgtgcggcaactgtcgaatccaacgacaatatgcagttcaacactaaagacatccaagtaagcaaagcttgtaaggaattcaccatcaccctgaaacacaccggtacccaacctaaaaccagcatgggtcacaacattgtcatcggtaaaactgaagacatggacggtattttcaaagatggcgttggcgcagctgacactgactacgttaaacctgacgatgcgcgcgttgttgcccacaccaaactgatcggcggcggcgaagagtcttccctgactctagatcctgccaaattggctgacggcgactacaaatttgcctgcaccttcccgggtcacggtgctttgatgaacggtaaaattactttggttgactaa

SEQ ID NO: 12 - protein, 183 aa, N.meningitidis
MKAYLALISAAVIGLAACSQEPAAPAAEATPAAEAPASEAPAAEAAPADAAEAPAAGNCAATVESNDNMQFNTKDIQVSKACKEFTITLKHTGTQPKTSMGHNIVIGKTEDMDGIFKDGVGAADTDYVKPDDARVVAHTKLIGGGEESSLTLDPAKLADGDYKFACTFPGHGALMNGKITLVD

SEQ ID NO: 13 - protein, 18 aa, N.meningitidis
SQEPAAPAAEATPAAEAP

SEQ ID NO: 14 - protein, 18 aa, N.meningitidis
SQEPAAPAAEATPAAEAP

(19) United States
(12) Patent Application Publication
(10) Pub. No.: US 2016/0243222 A1
(43) Pub. Date: Aug. 25, 2016

(54) NOVEL COMPOSITIONS AND ADJUVANTS

(71) Applicant: ID Biomedical Corporation of Quebec, Laval (CA)

(72) Inventors: Martin GAGNE, Laval (CA); Daniel LAROCQUE, Laval (CA); Martin PLANTE, Laval (CA)

(21) Appl. No.: 15/150,763

(22) Filed: May 10, 2016

Related U.S. Application Data
(63) Continuation of application No. 13/061,841, filed on Mar. 2, 2011.
(60) Provisional application No. 61/094,572, filed on Sep. 5, 2008.

(51) Int. Cl. A61K 39/39; A61K 39/095

(52) U.S. Cl. CPC A61K 39/39; A61K 39/095; A61K 2039/55516

(57) ABSTRACT

The present invention is directed toward adjuvants that effect an innate and/or a specific immune response. The adjuvants contain at least one lipoprotein, such as Lip, Lip fragments or Lip variants, where the lipoprotein comprises at least one pentameric unit and at least one lipid moiety. Adjuvants wherein the lipoprotein make up at least 10% of the adjuvant by weight/volume are provided.

### BACKGROUND

Immunization with many antigens more particularly soluble proteins results in the induction of weak responses or B cell and/or T cell tolerance whereas immunization with soluble proteins mixed with adjuvants tends to results in immunity. Many adjuvants contain microbial products that are known to have immunomodulatory properties. Heat-killed mycobacteria present in CFA, lipopolysaccharides (LPS), muramyl dipeptide, and bacterial toxins such as pertussis toxins, cholera toxin and E. coli enterotoxin have such properties. Bacterial porins are another class of bacterial components having immunomodulatory properties, in that particular case through the activation of the NF-κB pathway via the Toll-Like receptor 2 (TLR2).

A gonoccoccal homolog of the meningococcal Lip protein has been shown to stimulate cytokine release and NF-κB activation in epithelial cells in a Toll-Like Receptor 2 dependent manner. Fisette et al., 2003, JBC, Vol. 47, pp 46252. Specifically, Fisette, et al. demonstrated that triacylated Lip peptide, Pam3Cys—GGEKAAEAPAAEAS (SEQ ID NO: 1) also referred to as Lip peptide F62, can stimulate the production of inflammatory cytokines. The production of proinflammatory cytokines is linked to the capacity of adjuvants to improve immunogenicity of antigens. Proinflammatory cytokines have been shown to promote local inflammatory responses at site of microbial infection and mediate adherence of leukocytes to endothelial tissues and their transmigration by upregulation of adhesion molecules P-selectins, E-selectins, ICAMS, VCAMS (Reviewed by Henderson et al., 1996. Microbiol Rev. 60:316-341). Exposure to TNF-α and IL-1 (and other inflammatory mediators) at site of local inflammation can also influence the capacity of dendritic cells to mature, migrate to T-cell areas of lymphoid tissues, and present antigens. TNF-α which is one of the best-known proinflammatory cytokines can induce the production of IL-1 which in turn; promotes T cell-dependent antibody response in vivo and abrogates immunologic tolerance, induces expression of the IL-2 receptor and can enhance proliferation of CD4⁺ T cell clones. IL-1 could also increase permeability of mucosal barriers (Coyne, et al., 2002. Mol. Bio. Cell (9)3218-3234). In general, proinflammatory cytokines can activate T and B lymphocytes and IL-1 is a potent stimulator of hematopoiesis (Dinarello, 1994. Adv Pharmacol. 25:21-51).

Although, several adjuvant candidates have been evaluated over the years, for safety concerns, only a few have been approved and are available for human use so there is still an unmet need for new potent and safe adjuvants and immunostimulatory compositions for the purpose of enhancing innate immunity as well as increasing the potency of vaccines. Thus, the present invention provides new compositions, adjuvants, immunogenic compositions and vaccines.

### BRIEF DESCRIPTION OF THE DRAWINGS

FIG. 1: Western immunoblot showing the presence of the meningococcal (strain 8047) Lip protein in three different Proteosome preparations (V1 proteosome, V2 proteosome and Protollin) from Neisseria meningitidis.

FIG. 2: SDS-PAGE and Silver stain analysis of the purified meningococcal (strain 8047) Lip protein.

FIG. 3: In vitro NF-κB assay results showing that Proteosome adjuvant and Lip peptide lipidated MC58 (SEQ ID NO:9) are mainly TLR1 and 2 agonists.

FIG. 4: Inhibition assay results confirming the involvement of TLR2 in the NF-κB pathway activation by Lip peptide lipidated MC58 (SEQ ID NO:9).

FIG. 5: Dose-response activation of the NF-κB pathway by purified Lip protein preparations (SEQ ID NO:12).

FIG. 6: Influenza-specific serum IgG response in C57BL/6 mice following nasal instillation with 3 μg of SFV (A/Anti New Caledonia H1N1) combined with 10 μg of synthetic Lip peptide lipidated MC58 (SEQ ID NO:9).

FIG. 7: Immunogenicity of a SFV (3 μg) in wild-type C57BL/6 mice when co-instilled with increasing doses of purified Lip Protein (SEQ ID NO:12) preparations (prep No. 1).

FIG. 8: Immunogenicity of a SFV (3 μg) in wild-type C57BL/6 mice when co-instilled with increasing doses of purified Lip Protein (SEQ ID NO:12) preparations (prep No. 2).

### SUMMARY OF THE INVENTION

In one embodiment of the present invention, adjuvants comprising at least one lipoprotein wherein said lipoprotein comprises at least one first pentameric unit and wherein said lipoprotein makes up at least 10% of said adjuvant by weight/volume are provided.

In another embodiment, compositions are provided comprising at least one fragment and/or variant of Lip wherein said fragment and/or variant of Lip is a lipoprotein comprising at least one first pentameric unit and at least one lipid moiety.

In addition, immunogenic compositions are described comprising at least one adjuvant comprising at least one lipoprotein wherein said lipoprotein comprises at least one first pentameric unit wherein said lipoprotein makes up at least 10% of said adjuvant by weight/volume and at least one antigen.

Also provided herein are methods for making and using the adjuvants, compositions and immunogenic compositions described herein, including, but not limited to, methods of making and using vaccines and methods of inducing an immunoresponse in a mammal including a human.

### DETAILED DESCRIPTION OF THE INVENTION

An “immunogenic composition” as used herein refers to any one or more compounds or agents or immunogens capable of priming, potentiating, activating, eliciting, stimulating, augmenting, boosting, amplifying, or enhancing an adaptive (specific) immune response, which may be cellular (T cell) or humoral (B cell), or a combination thereof. Preferably, the adaptive immune response is protective, which may include neutralization of a virus (decreasing or eliminating virus infectivity) or other type of immune functional activity.

An “antigen” refers to a compound or composition capable of eliciting a cellular and/or humoral immune response, either alone or in combination or linked or fused to another substance. An antigen can be a peptide, polypeptide or protein of at least about 5 amino acids, a peptide of 10 amino acids in length, a fragment 15 amino acids in length, a fragment 20 amino acids in length or greater. An antigenic fragment of a protein can be isolated from a whole protein or it can be made synthetically and/or recombinantly. The antigen can comprise a “carrier” polypeptide and a hapten, e.g., a fusion protein or a carrier polypeptide fused or linked (chemically or otherwise) to another composition (described below). The antigen can be recombinantly expressed in an immunization vector, which can be simply naked DNA comprising the antigen's coding sequence operably linked to a promoter, e.g., a simple expression cassette.

As used herein “microorganism” includes but is not limited to any bacteria, virus, parasite, or prion found in nature. Antigens can be derived from either part or all of a microorganism. For example, an antigen derived from a microorganism may include, but is not limited to, a polypeptide present on the exterior of the microorganism. The polypeptide from said microorganism may be genetically or chemically fused to a second polypeptide, which may be endogenous or exogenous to said microorganism.

As used herein “allergen” means any immunogenic compound or organism or derivative, variant or fragment thereof capable of eliciting an allergic response in a mammal, including a human. Examples of allergens include, but are not limited to, antigens derived from house dust mites, Grass pollen, Ragweed pollen, cats, trees, molds and foods.

As used herein “lipoprotein” refers to any composition that comprises at least one protein and at least one lipid. The lipid or their derivatives may be covalently or non-covalently bound to the proteins. An example of a lipoprotein is Lip protein which may be isolated from various strains of bacteria, including but not limited to, Neisseria meningitidis.

As used herein “lipid” refers to any of a group of organic compounds, including the fats, oils, waxes, sterols, and triglycerides, that are insoluble in water but soluble in nonpolar organic solvents, are oily to the touch, and together with carbohydrates and proteins constitute the principal structural material of living cells. Lipids of the present invention include, but are not limited to, palmytoyl, phosphatidylethanolamine (PE), phosphatidylglycol (PG), phosphatidylcholine (PC), phosphatidylserine (PS), phosphatidylinositol (PI), and cardiolipin (CL). “Lipidated” refers to a compound such as a protein or polypeptide to which at least one lipid is associated. Lipids may be associated to a polypeptide or protein either covalently or non-covalently.

As used herein an “adjuvant” refers to any composition capable of acting as an immunostimulant or immunomodulator. Adjuvants of the present invention, when administered together with an antigen induce an innate immunity, that lead to the development of an adaptive immune response may by of a Th1-type and/or a Th2-type response to that antigen. Th1 cells typically produce IFN-γ, IL-2 and TNF-α and mediate cellular immunity to a large number of pathogens, particularly intracellular pathogens (McGuirk P and Mills K H. Trends Immunol 2002; 23(9): 450-5). Th2 cells produce generally IL-4, IL-5 and IL-13, favour humoral responses and are crucial to mount effective immune responses against helminth infections and many extracellular bacteria (Whary M T and Fox J G., Curr Top Med Chem 2004; 4(5): 531-8.). Adjuvants of the present invention may elicit an innate immune response or an adaptive response, the latter being directed against at least one specific antigen when administered to a mammal.

As used herein “innate immune response” refers to a response wherein a host produces immune cells and/or mechanism that defend a host from infection by other organisms, in a non-specific manner. During innate immune response, the cells of the innate system recognize, and respond to, pathogens in a generic way, but unlike the adaptive immune system, it usually does not confer long-lasting protective immunity or immune memory to the host. Innate immune responses provide immediate defense against infection, and are found in all classes of plant and animal life.

As used herein “weight/volume” refers to a percentage of a component of a composition over a given volume of the composition.

Outer membrane protein-based immunostimulatory compositions also referred to as “proteosome” have been described in the past (see, e.g., Lowell et al., J. Exp. Med. 167:658, 1988; Lowell et al., Science 240:800, 1988; Lynch et al., Biophys. J. 45:104, 1984; Lowell, in “New Generation Vaccines” 2nd ed., Marcel Dekker, Inc., New York, Basil, Hong Kong, page 193, 1997; U.S. Pat. No. 5,726,292; U.S. Pat. No. 4,707,543). Proteosomes may be prepared, for example, as described in the art (see, e.g., U.S. Pat. No. 5,726,292 or U.S. Pat. No. 5,985,284, incorporated herein by reference). Proteosome-based immunostimulatory compositions can be either co-instilled (usually intranasally) or formulated with antigens of various natures such as proteins, LPS and peptides. They have been shown to be very potent at inducing protective immunity against the influenza virus (Plante, et al. Vaccine. 2001; 20(1-2):218-25), the respiratory syncytial virus (Cyr, et al. Vaccine. 2007; 25(29):5378-89; Cyr, et al. Vaccine 2007; 25(16):3228-32.) and measles (Chabot et al. Vaccine. 2005; 23(11):1374-83.) in murine experimental models. In humans, proteosome delivery systems were very potent at promoting anti-LPS antibody responses in the context of a Proteosome-Shigella LPS vaccine. Strong protective immunity was also elicited in human against experimental influenza virus infection using an intranasal vaccine made of Proteosomes and subunit influenza vaccine (Langley et al. Vaccine. 2006; 24(10):1601-1608; Treanor J et al. Vaccine 2006; 24(3): 254-262.). In addition, Proteosome-formulated baculovirus-derived Influenza hemaglutinin (HA) given to mice intranasally elicits higher levels of HA-specific IgG2a and markedly reduced levels of IL-5 compared to mice given the antigen alone (Jones, et al. Vaccine 2003; 21(25-26): 3706-12).

Proteosome-based adjuvants are composed primarily of chemically extracted outer membrane proteins (OMPs) from Neisseria meningitidis (mostly porins A and B as well as class 4 OMP), maintained in solution by detergent (Lowell G H. Proteosomes for Improved Nasal, Oral, or Injectable Vaccines. In: Levine M M, Woodrow G C, Kaper J B, Cobon G S, eds, New Generation Vaccines. New York: Marcel Dekker, Inc. 1997; 193-206). Proteosomes can be formulated with a variety of antigens such as purified or recombinant proteins derived from viral or bacterial sources by diafiltration or traditional dialysis processes. Proteosome-based adjuvants may be prepared as described in the art (see, e.g., U.S. Pat. No. 5,726,292 or U.S. Pat. No. 5,985,284).

“Proteosome with LPS or Protollin” as used herein refers to preparations of proteosomes admixed (e.g., by the exogenous addition) with at least one kind of liposaccharide to provide a Proteosome-LPS composition (which can function as an immunostimulatory composition). Thus, the Proteosome-LPS composition can be comprised of two of the basic components of Protollin, which include (1) an outer membrane protein preparation of Proteosomes prepared from Gram-negative bacteria, such as Neisseria meningitidis, and (2) a preparation of one or more liposaccharides.

“Liposaccharide,” as used herein, refers to native (isolated or prepared synthetically with a native structure) or modified lipopolysaccharide or lipooligosaccharide. Liposaccharides may be endogenous to a first bacterium or may be derived from a second Gram-negative bacteria, such as Shigella flexneri or Plesiomonas shigelloides, or other Gram-negative bacteria (including Alcaligenes, Bacteroides, Bordetella, Borrellia, Brucella, Campylobacter, Chlamydia, Citrobacter, Edwardsiella, Ehrlicha, Enterobacter, Escherichia, Francisella, Fusobacterium, Gardnerella, Hemophilus, Helicobacter, Klebsiella, Legionella, Leptospira (including Leptospira interrogans), Moraxella, Morganella, Neiserria, Pasteurella, Proteus, Providencia, other Plesiomonas, Porphyromonas (including Porphyromonas gingivalis), Prevotella, Pseudomonas, Rickettsia, Salmonella, Serratia, other Shigella, Spirllum, Veillonella, Vibrio, or Yersinia species). Included within the definition of liposaccharide is both lipooligosaccharide (LOS), which is understood in the art to mean a liposaccharide having a glycan chain consisting of 10 or fewer monosaccharide subunits, and lipopolysaccharide (LPS), which is understood in the art to mean a liposaccharide having a glycan chain comprising more than 10 monosaccharide subunits. Thus, LOS and LPS may be endogenous or exogenous. A liposaccharide may be in a detoxified form (i.e., having the Lipid A core removed) or may be in a form that has not been detoxified. For example, an LPS that contains multiple lipid A species such as P. gingivalis LPS may be used in the compositions described herein (see, e.g., Darveau, et al., Infect. Immun. 72:5041-51 (2004)). The liposaccharide may be prepared, for example, as described in U.S. Patent Application Publication No. 2003/0044425.

Protollin should also be understood to optionally include lipids, glycolipids, glycoproteins, small molecules, or the like, and combinations thereof “Proteosome: LPS or Protollin” may be prepared, for example, as described in U.S. Patent Application Publication No. 2003/0044425, incorporated herein by reference. Proteosome-Shigella-flexneri 2a LPS complexes, known as Protollin, have been administered in Phase I and II clinical trials as a vaccine against dysentery, to more than 100 volunteers and were found to be safe and non-toxic (Fries, et al. Infect Immun 2001; 69(7): 4545-53.). Protollin was delivered at doses of up to 1.5 mg of LPS intranasally, without adverse events (Fries, et al. Infect Immun 2001; 69(7): 4545-53.); Jones, et al. Vaccine 2004; 22(27-28): 3691-7). Mouse immunization with Protollin combined with a recombinant plague antigen, F1-V (capsular and virulence associated proteins, respectively) enhanced the release of TNF-α, while concurrently suppressing secretion of the regulatory cytokine IL-10 compared to the F1-V alone (Jones, et al. Vaccine 2005). Finally, Protollin given with a split measles virus antigen skews the IgG1/IgG2a ratio towards Th1-biased responses (Chabot, et al. Vaccine 2005; 23(11): 1374-83).

As used herein “pentameric unit” refers to any five contiguous amino acids. A pentameric unit may form part of a larger polypeptide. More specifically, a pentameric unit includes, but is not limited to, five contiguous amino acids such as AAEAX (SEQ ID NO:7), wherein X can be any amino acid. Thus, a pentameric unit may include any or all of the following sequences: AAEAS (SEQ ID NO:4), AAEAA (SEQ ID NO:5), and AAEAP (SEQ ID NO:6).

As used herein a “neurological disease or disorder” refers to any condition involving a neuronal abnormality, including but not limited to, a neurodegenerative disease or disorder. For instance, neurodegenerative diseases and disorders are neurological disease characterized by destruction or deterioration of selective neuronal and/or myelin populations. Exemplary neurodegenerative diseases include, but are not limited to, acute diseases such as stroke (ischemic or haemorrhagic), traumatic brain injury and spinal cord injury as well as chronic diseases including Alzheimer's disease, fronto-temporal dementias (tauopathies), peripheral neuropathy, Parkinsonian syndromes such as Parkinson's disease (PD), Creutzfeldt-Jakob disease (CJD), Prion diseases, Schizophrenia, amyotrophic lateral sclerosis (ALS), multiple sclerosis, cerebral amyloid angiopathy (CAA), Huntington's disease, inclusion body myositis. and mild cognitive impairment (MCI). Neurodegenerative disease is associated with progressive nervous system dysfunction, and often leads to atrophy of affected central or peripheral nervous system structures.

Alzheimer's disease (AD) is a β-amyloid associated disease which is a progressive neurodegenerative disorder that is the predominant cause of dementia in people over 65 years of age. AD is characterized by massive neuronal cell loss in certain brain areas, and by the deposition of proteinaceous material in the brains of AD patients. These deposits contain neurofibrillary tangles and β-amyloid plaques. The major protein component of the β-amyloid plaque is Aβ. Increased accumulation of Aβ has been postulated to significantly contribute to the pathogenesis of AD, and is also associated with various other amyloidoses and neurological disorders also referred to herein as “β-amyloid associated disease,” such as Parkinson's disease, Down syndrome, diffuse Lewy body disease, progressive supranuclear palsy, and Hereditary Cerebral Hemorrhage with Amyloidosis-Dutch Type (HCHWA-D), cerebral amyloid angiopathy (CAA), and mild cognitive impairment (MCI).

The present invention demonstrates that purified Lip protein and derived lipopeptides from Neisseria species act as a strong innate immune activator, acting through specific Toll like receptors. Intranasal immunization using split influenza virus vaccine (SFV; A/New Caledonia/20/99 (H1N1)) co-instilled with the purified Lip protein was shown to elicit influenza-specific serum IgG levels significantly higher than the SFV alone (in C57BL/6 mice).

Thus, the present invention provides adjuvants comprising at least one lipoprotein wherein said lipoprotein comprises at least one first pentameric unit and wherein said lipoprotein makes up at least 10% of said adjuvant by weight/volume. In some aspects, the lipoprotein is Lip protein and/or a fragment and/or a variant thereof. Lip protein and/or a fragment and/or a variant thereof can be triacylated lipopeptidic fragment (Pam3CysLip) and/or diacylated lipopeptidic fragment (Pam2CysLip) and others lipopeptide fragment from Neisseria sources. Lip can be isolated from Neisseria meningitidis or other Neisseria species. The neisserial strain may be selected from the species consisting of: N. gonorrhoeae, N. meningitidis, N. lactamica and N. cinerea. Other Neisseria such as, but not limited to, Neisseria polysaccharea could also be used. In some aspects, the Lip protein is isolated from Neisseria meningitidis. Neisseria meningitidis may be a B strain. In some aspects, Lip may be isolated from Neisseria meningitidis which is strain 8047. Lip proteins of the present invention may be encoded by a polynucleotide comprising SEQ ID NO:11. Lip proteins of the present invention may comprise SEQ ID NO:12. Fragments of Lip proteins of the present invention may comprise SEQ ID NO:13.

As is understood in the art, Lip proteins of the present invention may be isolated wild type protein from any of the various bacteria or homologues of bacteria described herein. In addition, Lip proteins of the present invention may be produced recombinantly and/or overexpressed in a host cell. Recombinantly expressed Lip protein and/or fragments and/or variants thereof, may be prepared by processes well known in those skilled in the art from genetically engineered host cells comprising expression systems. Accordingly, in a further aspect, the present invention relates to expression systems that comprise a polynucleotide or polynucleotides of the present invention, to host cells which are genetically engineered with such expression system, and to the production of polypeptides of the invention by recombinant techniques.

For recombinant production of the polypeptides of the invention, host cells can be genetically engineered to incorporate expression systems or portions thereof or polynucleotides of the invention. Introduction of a polynucleotide into the host cell can be effected by methods described in many standard laboratory manuals, such as Davis, et al., BASIC METHODS IN MOLECULAR BIOLOGY, (1986) and Sambrook, et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as but not limited to cells of streptococci, staphylococci, enterococci, E. coli, Streptomyces, cyanobacteria, Bacillus subtilis, Moraxella catarrhalis, Haemophilus influenzae and Neisseria meningitidis; fungal cells, such as cells of a yeast, Kluveromyces, Saccharomyces, a basidiomycete, Candida albicans and Aspergillus; insect cells such as cells of Drosophila S2 and Spodoptera Sf9; animal cells such as CHO, COS, HeLa, C127, 3T3, BHK, 293, CV-1 and Bowes melanoma cells; and plant cells, such as cells of a gymnosperm or angiosperm.

A great variety of expression systems can be used to produce the polypeptides of the invention. Such vectors include, among others, chromosomal-, episomal- and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, picornaviruses, retroviruses, and alphaviruses and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL

In yet another aspect of the invention, the lipoprotein is a fragment of Lip. In one aspect, the fragment of Lip comprises an H.8 peptide (Woods J P, Aho E L, Barritt D S, Black J R, Connell T D, Kawula T H, Spinola S M, Cannon J G. 1987. The H8 antigen of pathogenic Neisseriae. Antonie Van Leeuwenhoek. 1987; 53(6):533-6; Fisette et al., 2003). As is understood in the art, the outer membrane antigen of N. gonnorhoeae can be described by the following 88 amino acid sequence (SEQ ID NO:2) and contains the H.8 epitope underlined therein (CGGEKAAEAPAAEAS SEQ ID NO:3):

(SEQ ID NO: 2)
MKKSLFAAAL LSLALAACGG EKAAEAPAAE ASSTEAPAAE
10 20 30
APAAEAPAAE AAAAEAPAAE APAAEAPAAE AAATEAPAAE
40 50 60 70
APAAEAAK
80

As is also understood in the art, fragment of the Lip protein comprising the H.8 epitope is highly conserved among the Neisseria genus. The N-terminal of SEQ ID NO: 2 can be considered amino acids 1-50 or amino acids 1-40 or amino acids 1-35 of SEQ ID NO:2. In another embodiment of the present invention, the fragment of Lip may comprise at least one additional amino acid at the N-terminal or C-terminal of said H.8 peptide.

In one aspect of the present invention, lipoprotein comprises at least one lipid. Lipoproteins of the present invention may comprise at least one lipid moiety selected (but not limited to) from the group of: palmytoyl, phosphatidylethanolamine (PE), phosphatidylglycol (PG), phosphatidylcholine (PC), phosphatidylserine (PS), phosphatidylinositol (PI), and cardiolipin (CL).

In another aspect of the present invention, the lipoprotein comprises a first pentameric unit. This first pentameric unit may be selected from the group of: AAEAS (SEQ ID NO:4), AAEAA (SEQ ID NO:5), and AAEAP (SEQ ID NO:6). In another aspect, the lipoprotein further comprises a second pentameric unit. The second pentameric unit may be selected from the group of: AAEAS (SEQ ID NO:4), AAEAA (SEQ ID NO:5), and AAEAP (SEQ ID NO:6). In some aspects, the second pentameric unit is the same as said first pentameric unit. In yet another aspect, the second pentameric unit is different than said first pentameric unit. Pentameric units may be contiguous within the amino acid sequence of the lipoprotein or they may be separated by one or more amino acids. The adjuvants of the present invention may comprise at least one lipoprotein which is a recombinant protein. Additionally, the adjuvants of the present invention may comprise at least one lipoprotein which is synthetic.

In another aspect, the adjuvants of the invention are capable of acting via innate immune receptors such as those of the TLR family. In some aspects, the adjuvants of the present invention induce an innate immune response when administered to a mammal. The mammal may be human.

In some aspects the adjuvant further comprises at least one antigen. In some aspects the antigen comprises a fragment and/or variant and/or hybrid antigen from the group of: cancer antigen, influenza virus, Neisseria species, malarial parasite, HIV, birch pollen, DerP1, grass pollen, RSV, at least one β-amyloid antigen, at least one myelin antigen, and tuberculosis. More specifically, antigens may include, but are not limited to, at least one antigen from Neisseria meningitidis.

The adjuvants of the present invention can be administered by a route selected from: intramuscular, intravenous, mucosal, intra-cerebral, intra-spinal route subcutaneous, sublingual, transdermal, and inhalation. The mucosal route may be via the nasal, rectal, oropharyngeal, ocular or genitourinary mucosa. The adjuvants of the present invention may further comprise at least one excipient and/or pharmaceutically acceptable carrier.

The present invention further provides compositions comprising at least one fragment and/or variant of Lip wherein said fragment and/or variant of Lip is a lipoprotein comprising at least one first pentameric unit and at least one lipid moiety. At least one lipid moiety of these compositions may be selected from the group of: palmytoyl, phosphatidylethanolamine (PE), phosphatidylglycol (PG), phosphatidylcholine (PC), phosphatidylserine (PS), phosphatidylinositol (PI), and cardiolipin (CL). The lipoprotein may be a recombinant protein or synthetic. Compositions of the present invention may comprise Lip protein encoded by a polynucleotide comprising SEQ ID NO:11 or Lip protein that comprises SEQ ID NO:12. Additionally, composition of the present invention may comprise SEQ ID NO:8.

The first pentameric unit of a lipoprotein of the compositions of the present invention may be selected from the group of: AAEAX (SEQ ID NO:7), wherein X can be any amino acid. In some aspects, the first pentameric unit is selected from the group of: AAEAS (SEQ ID NO:4), AAEAA (SEQ ID NO:5), and AAEAP (SEQ ID NO:6). In another aspect, the lipoprotein of the compositions further comprises a second pentameric unit. The second pentameric unit may be selected from the group of: AAEAX, wherein X can be any amino acid. In some aspects, the second pentameric unit is selected from the group of: AAEAS (SEQ ID NO:4), AAEAA (SEQ ID NO:5), and AAEAP (SEQ ID NO:6). In some aspects, the second pentameric unit is the same as said first pentameric unit. In some aspects, the second pentameric unit is different that the first penatmeric unit. Compositions of the present invention may be capable of acting as an adjuvant. When compositions of the present invention act as an adjuvant they may act via an innate immune receptor and may induce an immune response when administered to a mammal. The may act as a TLR1 and/or a TLR2 and/or a TLR4 agonist.

The composition of the present invention may comprise at least one antigen. The antigen may comprise a fragment and/or variant and/or hybrid antigen from the group of: cancer antigen, influenza virus, Neisseria species, malarial parasite, HIV, birch pollen, DerP1, grass pollen, RSV, at least one β-amyloid antigen, at least one myelin antigen, and tuberculosis. The compositions of the present invention may also comprise β-amyloid and/or a fragment and/or a variant and/or a fusion thereof. More specifically, antigens may include, but are not limited to, at least one antigen from Neisseria meningitidis. Compositions can be administered by a route selected from: intramuscular, intravenous, mucosal, intra-cerebral, intra-spinal, subcutaneous, sublingual, transdermal and inhalation. The mucosal route may be via the nasal, rectal, oropharyngeal, ocular or genitourinary mucosa. The compositions of the present invention may comprise at least one excipient or pharmaceutically acceptable carrier.

Also provided with the present invention are methods of treating a human suffering form a neurological disease or disorder comprising administering any one of the adjuvants, compositions, immunogenic compositions and vaccines of the present invention, either alone or in combination. In some instances, the neurological disease may be a fl-amyloid associated disease. The fl-amyloid associated disease may include, but is not limited to, Alzheimer's disease. In another aspect, the adjuvants and compositions of the present invention may be combined with at least one Myelin antigen. In some instances the compositions may be used to promote spinal cord regeneration or Multiple sclerosis treatment or fl-amyloid immunization.

Also provided in the present invention are immunogenic compositions comprising at least one adjuvant comprising at least one lipoprotein wherein said lipoprotein comprises at least one first pentameric unit wherein said lipoprotein makes up at least 10% of said adjuvant by weight/volume and at least one antigen. In some aspects at least one antigen comprises a fragment and/or variant and/or hybrid antigen from the group of: cancer antigen, influenza virus, Neisseria species, malarial parasite, HIV, birch pollen, DerP1, grass pollen, RSV, at least one fl-amyloid antigen, at least one myelin antigen, and tuberculosis. Vaccines of the present invention can be administered by a route selected from: intramuscular, intravenous, intra-cerebral, intra-spinal, mucosal, subcutaneous, sublingual, transdermal, and inhalation. The mucosal route may be via the nasal, rectal, oropharyngeal, ocular or genitourinary mucosa.

Also provided are methods of making an adjuvant comprising:
optionally culturing at least one cell comprising at least one Lip and/or fragment and/or variant thereof;
optionally killing said at least one cell with heat to form a cell paste;
releasing from at least one cell a composition comprising at least one Lip and/or fragment and/or variant thereof from said at least one cell comprising contacting said at least one said cell with at least one agent capable of solubilizing at least one lipid and optionally an osmolalic agent, forming a mixture comprising said Lip and/or fragment and/or variant thereof and cell debris;
adding an agent capable of separating said cell debris from said Lip and/or fragment and/or variant thereof
separating said separated cell debris from said mixture;
removing said at least one agent capable of solubilizing at least one lipid and said Lip and/or fragment and/or variant thereof wherein said at least one Lip and/or fragment and/or variant thereof remains soluble; and
further purifying said Lip and/or fragment and/or variant thereof though at least one exchange columns.

The methods of the present inventions may further comprise cleaving said Lip and/or fragment and/or variant of thereof to form a first segment and second segment of said Lip and/or fragment and/or variant of thereof. In addition, the methods of the present invention may further comprise adding at least one excipient and/or pharmaceutical carrier to said adjuvant. Culture may be grown in animal free media.

In yet another aspect of the present invention, methods are provided for making a vaccine or immunogenic composition comprising admixing an adjuvant of the present invention with at least one antigen.

In another embodiment, methods are provided for eliciting an immunune response in a human comprising administering at least one composition of the present invention. In one aspect the composition is an adjuvant comprising at least one lipoprotein wherein said lipoprotein comprises at least one first pentameric unit and wherein said lipoprotein makes up at least 10% of said adjuvant by weight/volume. In another aspect the composition comprises at least one lipoprotein wherein said lipoprotein comprises at least one first pentameric unit and wherein said lipoprotein makes up at least 10% of said adjuvant by weight/volume. The immune response may be innate or adaptive. The adjuvant and or composition may further comprise an antigen.

In another aspect, Lip proteins encoded by a polynucleotide comprising SEQ ID NO:11 and Lip proteins comprising SEQ ID NO:12 are provided. In another aspect, lipoproteins comprising lipidated SEQ ID NO:13 is provided.

Vaccine preparation is generally described in New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

The amount of protein in each vaccine dose is selected as an amount which induces an immunoprotective response without significant, adverse side effects in typical vaccines. Such amount will vary depending upon which specific immunogen is employed and how it is presented. Generally, it is expected that each dose will comprise 1-1000 μg of protein, preferably 2-100 μg, most preferably 4-40 μg. An optimal amount for a particular vaccine can be ascertained by standard studies involving observation of appropriate immune responses in subjects. Following an initial vaccination, subjects may receive one or several booster immunization adequately spaced.

The formulations of the present invention maybe used for both prophylatic and therapeutic purposes. Accordingly in one aspect, the invention provides a method of treatment comprising administering an effective amount of a vaccine of the present invention to a patient.

The following examples illustrate various aspects of this invention. These examples do not limit the scope of this invention which is defined by the appended claims.

### EXAMPLES

### Example 1

### Detection of the Meningococcal Lip Protein Proteosome Preparations

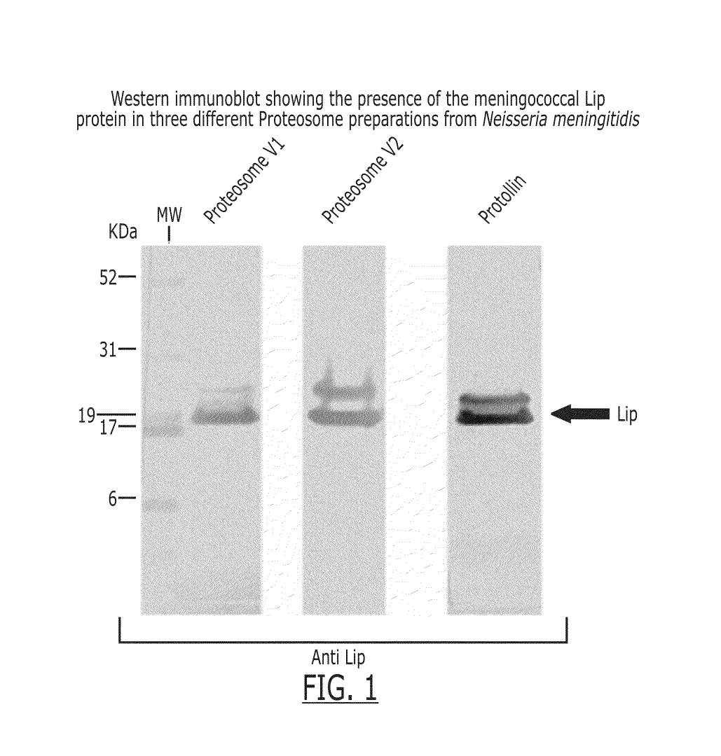

The presence of the meningococcal Lip protein was assessed in three different proteosome preparations by Western immunoblot. Briefly the Proteosome preparations were resolved by SDS-PAGE (BisTris 4-12% continuous gradient, 35 min migration at 200V) followed by transfer onto nitrocellulose membrane using the fast transfer system iBlot (invitrogen) for 7 minutes. Immunodetection of the Lip protein was performed using using mAb 2-1-CA2 (Provided by W. Zollinger) as primary antibody. Alkaline phosphatase coupled goat anti-mouse antibody was used as secondary antibody. The immunoblot was developed with the alkaline phosphatase substrate NBT/BCIP for 1 min at room temperature. As shown in FIG. 1, protein bands (doublet) in the 19-20 kDa apparent molecular weight range of the meningococcal Lip protein was clearly detected in both Proteosome (designated as V1 and V2 proteosomes) and Protollin (proteosome with LPS) preparations tested.

### Example 2

### V2 Proteosome Preparation

Lip protein was purified from Neisseria meningitidis (strain 8047) using the following process. Proteosome can be prepared by the following steps herein referred to as V2 Proteosome.

OMPs Extraction from the Whole Cells with a Zwitterionic Detergent

Outer membrane proteins from Heat-killed Neisseria meningitidis were solubilized using a zwitterionic detergent. Two hundred and fifty (250) grams of Neisseria meningitidis (Strain 8047) cell paste were thawed for 12-24 hours at 2-8° C. and suspended in 1M sodium acetate buffer pH 5.0. The diluted cell paste was then mechanically homogenized using an IKA Ultra-Turrax homogenizer on ice for 20-30 minutes. The homogenized solution was then further diluted with 1.5 vol Milli-Q water and homogenized for 20-30 minutes at room temperature. Subsequently, one suspension volume of 1M CaCl₂/6% LDB buffer was added and the suspension was homogenized for an additional 60 minutes at room temperature. Resulting cell paste was used for next ethanol precipitation step.

### 20% Ethanol Precipitation

After the initial cell paste solubilization, ethanol at 4° C. was slowly added to a final concentration of 20% v/v ethanol while homogenizing. For this step, a slow flow rate of ethanol addition, combined with efficient mixing was used so as to not create local high ethanol concentration in the suspension that might precipitate the proteins of interest. After ethanol addition, the suspension was homogenized for an additional 14-16 minutes at room temperature. The suspension was then clarified by pumping the mixture at a flux rate of 100 LMH on two 0.1 m² disposable and scalable POD depth filters (Millipore Cat. MCOHC01FS1). This filtration step retains cellular debris (high molecular weight proteins) and nucleic acid that were both precipitated with the ethanol. Filters were then immediately chased using an equal volume of In-House Chase buffer (0.08M sodium acetate, 0.4M CaCl₂, 20% EtOH and 0.1% LDB.

### 10× Concentration

The OMP-filtrate was concentrated 10× on a Pellicon Mini 0.1 m², 30 kDa ultrafiltration cassette (Millipore Cat. P2C030C01) at a flow rate of 330 ml/min and with a TMP manually adjusted at 10-11 psi. Afterwards, a micro-BCA assay (MTDV-0036, Rev.2) was performed on the 10× concentrate and the suspension is incubated at 2-8° C. overnight.

Diafiltration until [LDB]<200 ppm

On the second day (after the 10× concentration), the solution was highly concentrated in LDB detergent (6.3±0.1%) and contained undesirable impurities such as ethanol, sodium acetate and calcium chloride. A diafiltration was performed on the 10× retentate 1) to reduce the detergent concentration, 2) to lower undesirable impurities, 3) to remove the lower molecular weight proteins and finally, 4) to have the OMPs and the LOS in solution and in the final and human injectable PBS buffer.

The diafiltration was performed using another 30 kDa ultrafiltration cassette (Millipore Cat. P2C030C01) and two different buffers were used to diafilter the bulk at a constant volume. The first buffer was the TEN 1× buffer pH 8.0 (50 mM TrisBase, 10 mM EDTA and 150 mM NaCl) against which the suspension was diafiltered for 20 DV. Product was further diafiltered against the PBS Buffer at pH 7.4 (Gibco, Cat. 70011-044) until the LDB concentration was below 200 ppm, as determined by an On-line HPLC method (MTDV-0042). For this diafiltration step, it was important to diafilter first against the TEN buffer to remove calcium from the retentate before it comes in contact with PBS. This step avoided possible precipitate formation between the phosphate in the PBS buffer and the calcium from the calcium chloride buffer.

Concentration to 4.5 mg/ml and if Necessary, Diafiltration Until [LDB]<300 ppm

The suspension, which was below 200 ppm LDB, was concentrated using the same ultrafiltration cassette and set-up to 4.5 mg/ml using the micro-BCA assay result obtained on the Retentate after 10× concentration and considering a loss of 50%. After concentration, LDB concentration was measured. If LDB concentration was >300 ppm, the suspension was diafiltered against an additional volume of PBS buffer. If LDB concentration was lower than 300 ppm, no additional volume of PBS buffer was passed.

### Sterile Filtration

The final product was sterile filtered in the BioSafety Cabinet at a constant pressure of 50 psi and through two 0.22 sterilizing Grade and disposable Millipk-60 filter units (Millipore, Cat.MPGL066H2). Lip protein was purified from this sterile filtrate.

### Example 3

### Lip Protein Purification

Lip protein was purified from the Proteosome preparation of Example 2 as described below.

### Lip Purification

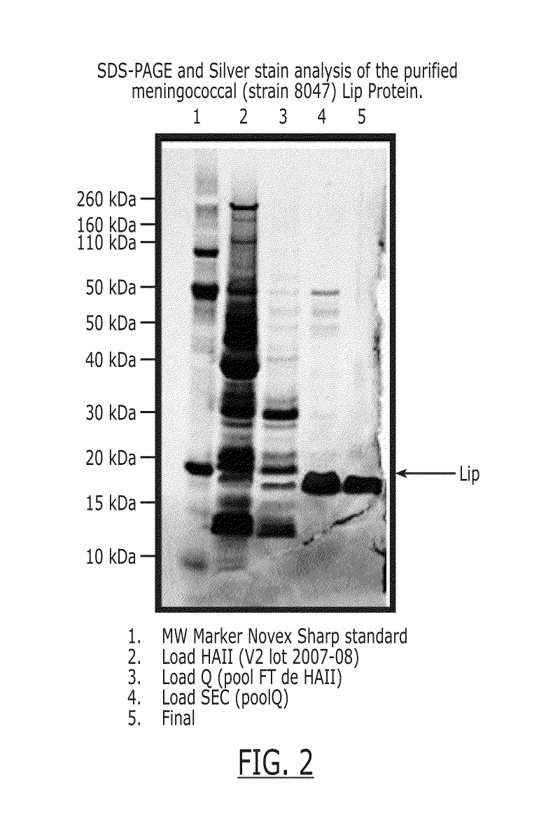

1. The sterile filtrate was diluted 10-fold in HAII loading buffer (20 mM sodium phosphate pH 7.0; 1 mM EDTA; 1% Empigen BB).
   The resulting solution was loaded on a hydroxylapatite type II column (Bio-Rad) previously equilibrated with HAII loading buffer. Only impurities bind on the column and Lip protein was found in the flow through. Flow through was harvested and used as a load for the second step.
   Silver stained SDS PAGE and western blot were performed to identify positive fractions.
2. HAII flow through containing the Lip protein was diluted 2 fold in Q loading buffer (20 mM Tris pH 8.5; 1 mM EDTA; 1% Empigen BB). Tris concentration was adjusted to 20 mM with Tris base powder and pH is adjusted to 8.5.
   The resulting solution was loaded on a Q sepharose HP column (GE Healthcare) previously equilibrated with Q loading buffer. Elution was performed by a gradient of increasing salt concentration from 0 to 500 mM NaCl in 8 column volumes followed by a gradient from 500 mM to 1M NaCl in 2 column volumes. Lip protein was eluted from the column at approximately 200 mM NaCl as identified by silver stained SDS PAGE and western blot.
3. Positive fractions for Lip protein were pooled and the resulting solution concentrated on a centricon (Millipore) to a volume of 9 ml, to allow optimal resolution by size exclusion chromatography (SEC).
   Concentrated Q eluate was loaded on a Superdex 75 2660 Hi load column (GE Healthcare) previously equilibrated with SEC buffer (20 mM Tris pH8.5; 1 mM EDTA; 150 mM NaCl; 1% Empigen BB) to remove high molecular weight contaminants.
   High Empigen BB concentration was maintained at this step to prevent aggregation of Lip protein with contaminants.
   Fractions containing pure Lip protein were pooled after identification by silver stained SDS PAGE and western blot.
4. Decreasing of Empigen BB concentration was performed by Q sepharose FF chromatography. Pool of the Lip positive SEC fractions was diluted 10 fold in Q loading buffer (20 mM Tris pH 8.5; 1 mM EDTA; 1% Empigen BB).
   Resulting solution was loaded on a Q sepharose FF column (GE Healthcare) previously equilibrated with Q loading buffer. After binding of Lip protein, column was washed with the same buffer containing 0.1% Empigen BB. Lip protein was eluted by a step of 200 mM NaCl in 0.1% Empigen BB.
   Positive fractions were pooled and kept at −20° C. The final Lip protein preparation was resolved by SDS-PAGE followed by silver staining as described in the SilverXpress kit (invitrogen) as shown in FIG. 2. The identity of the protein was also confirmed by Western Immunoblot using the 2-1-CA2 anti-Lip protein antibody. Extracted OMP's were passed through an hydroxyapatite Type II (HAII) column in negative mode (binding of impurities only) before being purified on a Qsepharose HP anion exchange column. A size ex clusion polishing step on superdex 75 was used to remove high molecular weight contaminants.

### Example 4

### Lip Peptide Fragment Amino Acid Sequence from N. gonorrhoeae (StrainF62)

As described previously H8 peptide is understood in the art to be part of the outer membrane antigen of N. gonnorhoeae F62 strain and can be described by the following 88 amino acid sequence (SEQ ID NO:2) and contains the H.8 epitope underlined therein (CGGEKAAEAPAAEAS SEQ ID NO:3):

(SEQ ID NO: 2)
MKKSLFAAAL LSLALAACGG EKAAEAPAAE ASSTEAPAAE
10 20 30
APAAEAPAAE AAAAEAPAAE APAAEAPAAE AAATEAPAAE
40 50 60 70
APAAEAAK
80

The H.8 epitope from N. gonnorhoeae (F62) can either be lipidated or non-lipidated as designated below:

Lipidated (F62):
(SEQ ID NO: 1)
Pam3Cys-GGEKAAEAPAAEAS
Non-lipidated (F62):
(SEQ ID NO: 3)
CGGEKAAEAPAAEAS

### Example 5

### Lip Protein and Fragment—Sequence of Peptide Synthesized from N. meningitidis

The corresponding polypeptide from N. meningitidis MC58 strain (NCBI Genbank number: NP_274531) was isolated and is presented below as SEQ ID NO:8.

| (SEQ ID NO: 8) | |
|---|---|
| 1 | MKKSLFAAAL LSLVLAACGG EKAAEAPAAE APAAEAPATE APAAEAPAAE APAAEAPAAE |
| 61 | AAATEAPAAE AAATEAPAAE AAATEAPAAE APAAEAAK |

The corresponding H.8 polypeptide from N. meningitidis MC58 strain (NCBI Genbank number: NP Non-lipidated: 274531) was synthetized and is presented below as SEQ ID NO:9 and 10 (lipidated MC58 and non-lipidated (NL) MC58). This sequence can be designated as either the lipidated or non-lipidated form.

Lipidated MC58:
(SEQ ID NO: 9)
Pam3Cys-GGEKAAEAPAAEAP
Non-Lipidated (NL) MC58:
(SEQ ID NO: 10)
CGGEKAAEAPAAEAP

### Example 6

### Lip Protein Isolated from Neisseria meningitidis (Strain 8047)

The DNA sequence from N. meningitidis (strain 8047) was sequenced and is presented as SEQ ID NO:11 below. Lip DNA sequence (coding strand) from Neisseria meningitidis (strain 8047):

(SEQ ID NO: 11)
ATG AAA GCG TAT CTG GCT CTG ATT TCT GCC GCC GTT
ATC GGT TTG GCT GCC TGC TCT CAA GAA CCT GCC GCG
CCT GCT GCC GAG GCA ACT CCT GCT GCT GAA GCA CCC
GCT TCC GAA GCG CCT GCC GCC GAA GCT GCT CCT GCA
GAT GCT GCC GAA GCC CCT GCT GCC GGC AAC TGT GCG
GCA ACT GTC GAA TCC AAC GAC AAT ATG CAG TTC AAC
ACT AAA GAC ATC CAA GTA AGC AAA GCT TGT AAG GAA
TTC ACC ATC ACC CTG AAA CAC ACC GGT ACC CAA CCT
AAA ACC AGC ATG GGT CAC AAC ATT GTC ATC GGT AAA
ACT GAA GAC ATG GAC GGT ATT TTC AAA GAT GGC GTT
GGC GCA GCT GAC ACT GAC TAC GTT AAA CCT GAC GAT
GCG CGC GTT GTT GCC CAC ACC AAA CTG ATC GGC GGC
GGC GAA GAG TCT TCC CTG ACT CTA GAT CCT GCC AAA
TTG GCT GAC GGC GAC TAC AAA TTT GCC TGC ACC TTC
CCG GGT CAC GGT GCT TTG ATG AAC GGT AAA ATT ACT
TTG GTT GAC TAA

The corresponding Lip protein sequence from Neisseria meningitidis (strain 8047) is presented below as SEQ ID NO:12

| (SEQ ID NO: 12) | |
|---|---|
| 1 | MKAYLALISA AVIGLAACSQ EPAAPAAEAT PAAEAPASEA PAAEAAPADA |
| 51 | AEAPAAGNCA ATVESNDNMQ FNTKDIQVSK ACKEFTITLK HTGTQPKTSM |
| 101 | GHNIVIGKTE DMDGIFKDGV GAADTDYVKP DDARVVAHTK LIGGGEESSL |
| 151 | TLDPAKLADG DYKFACTFPG HGALMNGKIT LVD |

Three pentameric units are underlined within SEQ ID NO:12.
The corresponding H8 polypeptide from N. meningitidis 8047 strain was isolated and is presented below as SEQ ID NO: 13 and 14, representing the lipidated form and non-lipidated form:

Lip peptide 8047:
(SEQ ID NO: 13)
Pam3Cys-SQ EPAAPAAEAT PAAEAP
Lip peptide 8047 NL:
(SEQ ID NO: 14)
SQ EPAAPAAEAT PAAEAP

### Example 7

### Proteosome-Based Adjuvants and Lipidated MC58 (SEQ ID NO:9) are TLR1+2 Agonist

Proteosome-based adjuvants may be prepared as described in the art (see, e.g., U.S. Pat. No. 5,726,292 or U.S. Pat. No. 5,985,284 (herein referred to as V1 proteosomes).

Proteosome-based adjuvants compositions with LPS may be prepared, for example, as described in U.S. Patent Application Publication No. 2003/0044425 (herein referred to as Protollin). Lipopeptides are present in a wide variety of microbes and stimulate immune responses through TLR1/2 or TLR2/6 heterodimers. The main receptor for lipopeptides is TLR2, which in combination with TLR1 recognises triacylated lipopeptides (such as Pam3Cys lipidated peptide), or in combination with TLR6 recognizes diacylated lipopeptides (such as Pam2Cys lipidated peptide) (Doyle and O'Neill, 2006). Innate immunity pathway components such as Toll-like receptors agonists are well known to activate NF-kB translocation and thereby activating promoter containing NF-kB binding elements (Janeway, C A, P Travers, M Walport, M J Shlomchik. Immunobiology, Book. New York: Garland Publishing, 2005. Akira, 2006. TLR signaling. Curr Top Microbiol Immunol. 2006; 311:1-16. Janeway C A Jr, Medzhitov R. 2002. Innate immune recognition. Annu Rev Immunol. 20:197-216). This TLR cell-based assay consist of a TLRs expressing cell line transfected with reporter constructs utilizing the NF-kB promoter element to screen cell media for such activity. These reporter cell lines were transfected with a plasmid expressing the secreted alkaline phosphatase, or “SEAP” reporter gene, as a consequence of NF-kB activation. The procedure is described herein: HEK293 cells stably expressing human TLR 1 and 2 or TLR4/MD2/CD14 (Invivogen, San Diego, Calif.) were cultured in 24-well plates in 500 DMEM supplemented with 10% heat-inactivated FBS in a 5% CO₂ incubator (DMEM media). At 80% confluence, cultures were transiently transfected with 500 ng/ml SEAP (secreted form of human embryonic alkaline phosphatase) reporter plasmid (pNifty2-Seap) (Invivogen) in the presence of lipofectamine 2000 (Invitrogen, Carlsbad, Calif.) in culture medium. Plasmid DNA and lipofectamine were diluted separately in serum-free medium and incubated at room temperature for 5 minutes. After incubation, the diluted DNA in lipofectamine-DMEM solution were mixed and the mixtures were incubated at room temperature for 20 minutes. Aliquots of 100 μl of the DNA/lipofectamine mixture containing 500 ng of plasmid DNA and lipofectamine were added on top of 400 μl of DMEM media of to each well of the cell culture plate, and the cultures were continued for 16 hours. After transfection, medium was replaced with fresh DMEM culture medium without serum, adjuvants were added to the cultures, and the cultures were continued for 5 hours. Transfected cells were exposed for 5 hours to either Proteosome, Lip proteins, Lip peptides and appropriate controls.

At the end of the treatment with the various agonists, 50 μl of culture supernatant was collected from each treatment and used for SEAP assay following manufacturer's protocol (Invivogen). Briefly, culture supernatants were incubated with QUANTI-Blue phosphate substrate (Invivogen) and the purple color generated was measured by a plate reader at 650 nm. The data are shown as the relative 650 nm optic density measure which reflects the NF-κB activity.

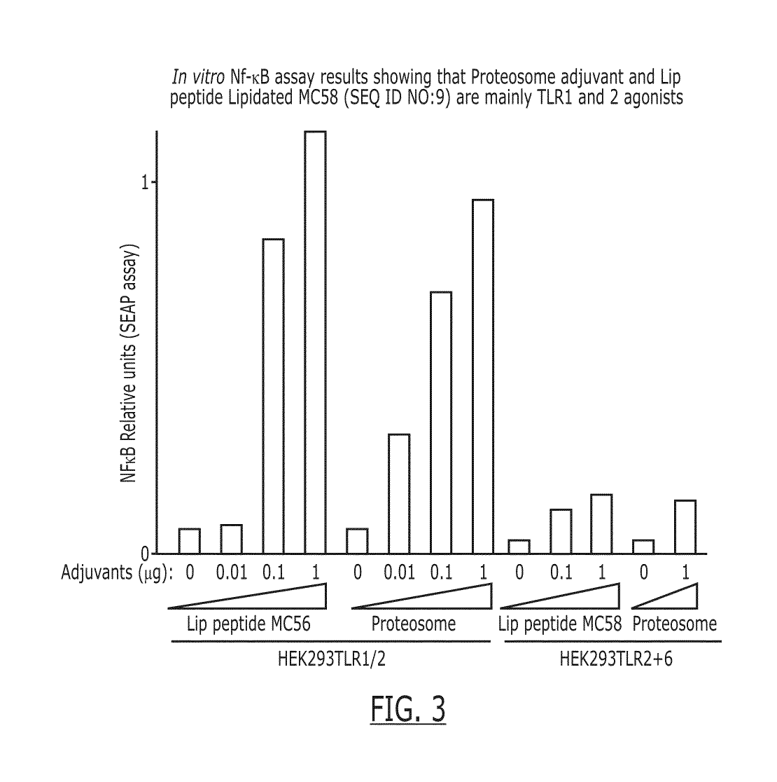

The effect of Proteosome, Lip peptides MC58 (SEQ ID NO: 9) and Lip protein (SEQ ID NO:12) on NF-kB activation in TLR1/2 or TLR2/6-expressing cell line was tested. Cells were exposed to Proteosome or Lip peptide MC58 (SEQ ID NO:9) at concentrations of 0 μg, 0.01 μg, 0.1 μg, and 1.0 μg each. The results of this study are shown in FIG. 3. NF-κB activation indicated that Lip peptide MC58 (SEQ ID NO:9) and Proteosome acted as a TLR1 and TLR2 agonist in a concentration dependent manner (see FIG. 3). These results indicate involvement of TLRs in vitro by SEQ ID NO:9 and suggest that Lip peptide and Lip protein are involved in activating an innate immune response.

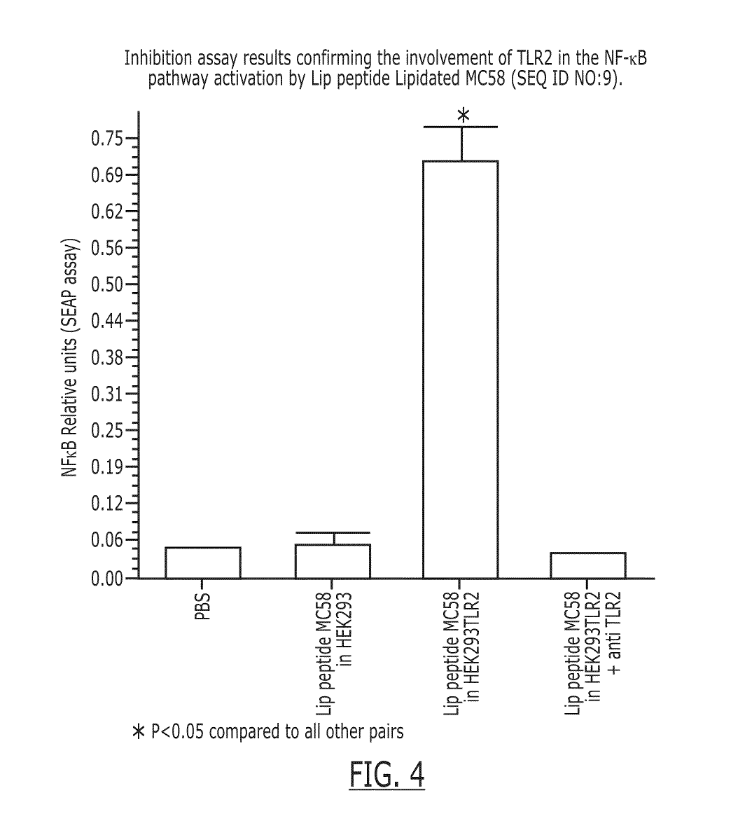

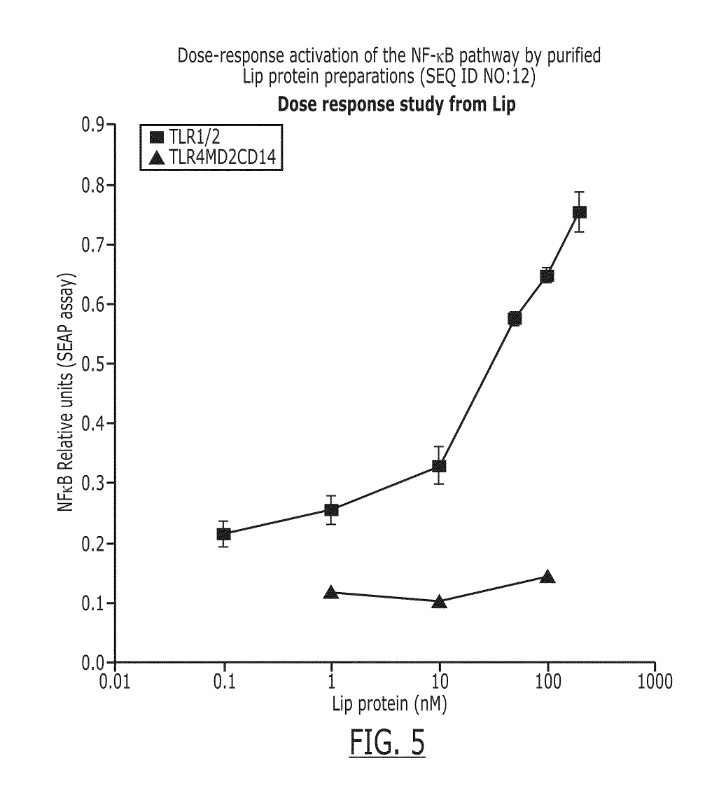

Upon specific inhibition of TLR2 by excess neutralizing anti-TLR2 antibodies, the ability of MC58 Lip peptide to stimulate TLR2-dependent NF-kB activation was abolished (FIG. 4). Moreover, control cells which do not contain TLR 2 did not respond showing that TLR2 is a receptor targeted by MC58 Lip peptide. A concentration-dependent response was observed in NF-kB activation in cells expressing TLR1/2 by purified Lip protein as shown in FIG. 5. In contrast, no significant increase of NF-κB activation was observed in cell line expressing TLR4/MD2/CD14, suggesting that Lip protein is a TLR1/2 agonist and not a TLR4 agonist. See FIG. 5. Dose-response activation of the NF-κB pathway by purified Lip protein preparations (SEQ ID NO:12).

### Example 8

### Adjuvant Properties of the Lipidated MC58 (SEQ ID NO:9) in Mice Using SFV as Model Antigen

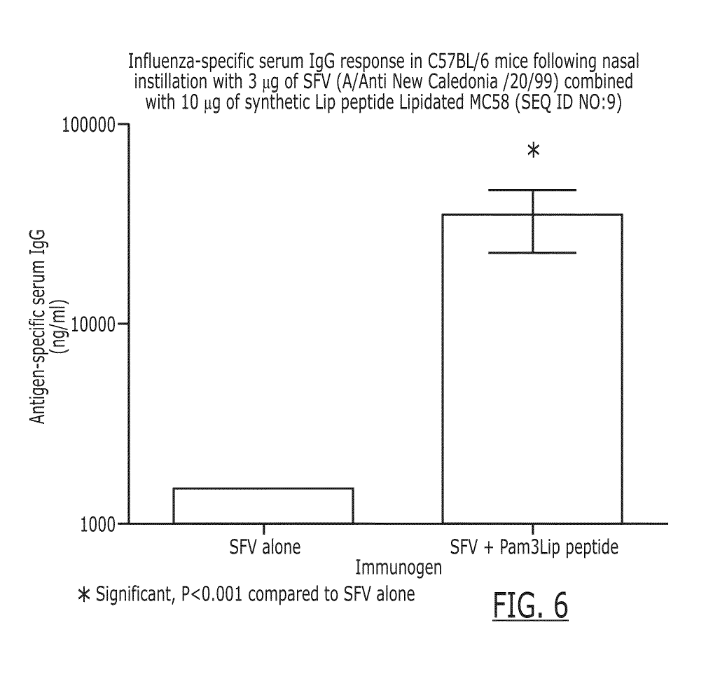

Groups of C57BL/6 mice were instilled (12.5 pt per nostril) nasally with 3 μg of SFV (A/New Caledonia/20/99) alone or in combination with 10 μg of synthetic Lip peptide MC58 (SEQ ID NO:9) under light isoflurane anesthesia on Day 0 and on Day 14. On day 28, mice were euthanized and exsanguinated by cardiac puncture. Influenza-specific IgG levels were measured in sera collected at Day 28 by ELISA using (A/New Caledonia/20/99) SFV as solid-phase antigen. Mice immunized nasally with SFV combined with 10 μg of synthetic Lip peptide MC58 (SEQ ID NO:9) were shown to develop statistically significant higher levels of serum influenza-specific IgG levels (see FIG. 6). This result clearly demonstrate the adjuvant properties of the synthetic Lip peptide MC 58 (SEQ ID NO:9) when administered by the nasal route.

### Example 9

### Evaluation of the of Lip Protein Adjuvant Potential (SEQ ID NO:12) in Mice Using SFV as Model Antigen (Antigen-Specific Antibody Response)

The capacity of the Lip protein isolated from N. meningitidis OMP preparation to increase immunogenicity of co-administered antigens was assessed in mice using SFV as model antigen. Groups of C57BL/6 mice were instilled nasally on Day 0 and Day 14 (under light anesthesia) with either 3 μg of split-flu vaccine (SFV) alone or SFV admixed with 0.35 μg or 1.0 μg purified Lip protein (SEQ ID NO:12). Mice were euthanized on day 28. Blood was collected upon euthanasia by cardiac puncture and serum samples frozen at −80° C. until ready for testing. Two similar studies were carried out using two different Lip protein preparations (named batch 1 and batch 2).

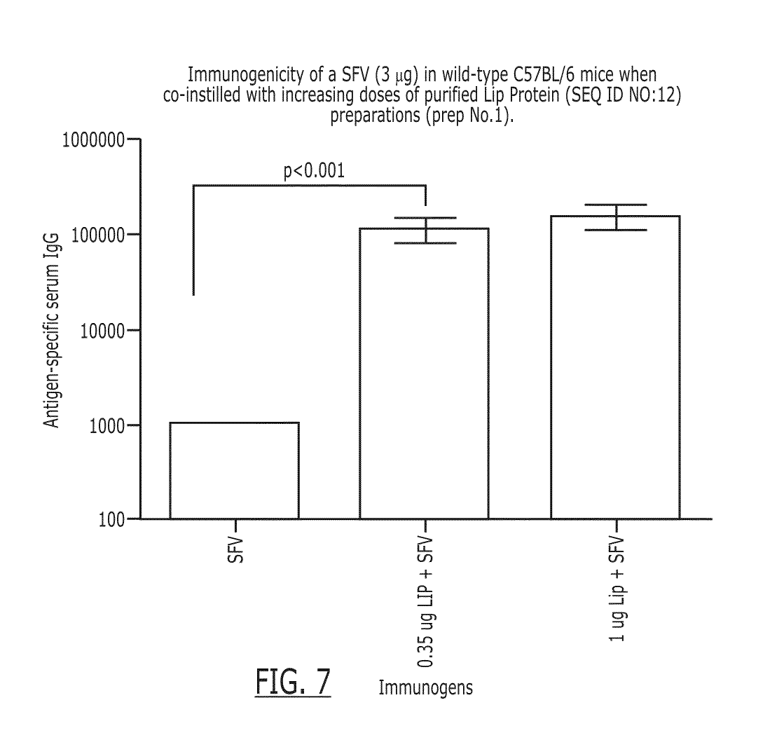

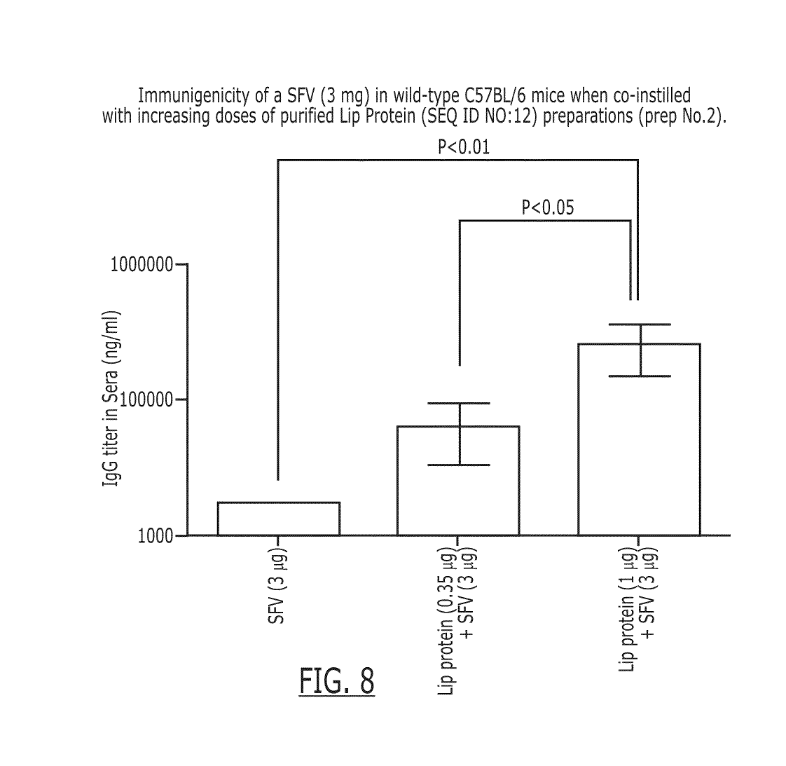

Antigen-specific antibody levels were measured by ELISA using homologous SFV as solid-phase antigen. In both studies performed (FIG. 7 and FIG. 8) statistically significant (p<0.001 and p<0.01) higher levels of antigen-specific IgGs were measured in sera collected from mice instilled with SFV admixed with any doses of Lip protein tested (SEQ ID NO:12). These results demonstrated the adjuvant properties of a purified Lip protein preparation for the elicitation of antibody response in the C57BL/6 mouse. Lip protein is able induce specific TLR signaling and suggest that a vaccine formulated with a composition comprising Lip protein or a fragment thereof could be used for mucosal immunization and vaccination.

What is claimed is:

1. An immunogenic composition comprising an adjuvant, said adjuvant comprising at least one lipoprotein comprising a lipid moiety and a polypeptide wherein the lipid moiety comprises at least one palmytoyl and the polypeptide is selected from the group of: SEQ ID NO: 9 and SEQ ID NO: 13.

2. The immunogenic composition of claim 1 further comprising an antigen.

3. The immunogenic composition of claim 2 wherein said antigen comprises a fragment and/or variant and/or hybrid antigen from the group of: cancer antigen, influenza virus, Neisseria species, malarial parasite, HIV, birch pollen, DerP1, grass pollen, RSV, at least one β-amyloid antigen, at least one myelin antigen, and tuberculosis.

4. The immunogenic composition of claim 1, wherein said adjuvant can be administered by a route selected from: rectal, intramuscular, intravenous, intraperitoneal, mucosal, enteral, parenteral, sublingual, transdermal, intra-cerebral, intra-spinal and inhalation.

5. The immunogenic composition of claim 4, wherein the mucosal route is via the nasal, oropharyngeal, ocular or genitourinary mucosa.

6. The immunogenic composition of claim 1, further comprising at least one excipient and/or pharmaceutically acceptable carrier.

7. The immunogenic composition of claim 1, wherein said adjuvant induces an immune response when administered to a human.

8. The immunogenic composition of claim 1, wherein said adjuvant induces an innate immune response when administered to a human.

9. The immunogenic composition of claim 1, wherein said lipoprotein is a recombinant protein.

10. The immunogenic composition of claim 1, wherein said lipoprotein is synthetic.

11. An immunogenic composition consisting of Pam3Cys—GGEKAAEAPAAEAP (SEQ ID NO 9).

12. An immunogenic composition consisting of Pam3Cys-SQ EPAAPAAEAT PAAEAP (SEQ ID NO 13).
